(19) United States
(12) Patent Application Publication
(10) Pub. No.: US 2004/0024260 A1
(43) Pub. Date: Feb. 5, 2004

(54) Process for preparing acetals and ketals with the aid of multistage pervaporation or vapor permeation

(75) Inventors: Hermann Winkler, Marl (DE); Heinz-Guenter Poll, Marl (DE); Manfred Neumann, Marl (DE); Guenther Koehler, Marl (DE); Frank Bauer, Haltern (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/423,929

(22) Filed: Apr. 28, 2003

(30) Foreign Application Priority Data
Apr. 27, 2002 (DE) 102 18 916.1

(51) Int. Cl. C07C 43/30; C07C 43/32

(52) U.S. Cl. 568/591; 568/595

(57) ABSTRACT

An acetal or a ketal are prepared by reacting a carbonyl compound and an alcohol in the presence of an acid catalyst and water. Water and alcohol are removed from the reaction mixture in an at least two-stage pervaporation or vapor permeation process using a plurality of membranes. Additional alcohol is fed into a retentate stream of at least one of the two stages. At least one of the plurality of membranes in the two-stage pervaporation or vapor permeation process is different from the other membranes in terms of selectivity behavior in the removal of water and alcohol.

### BACKGROUND OF THE INVENTION

1. Field of the Invention

The invention relates to a process for preparing an acetal and/or a ketal from a carbonyl compound and an alcohol in the presence of an acid catalyst, with water and an alcohol being separated off by multistage pervaporation or vapor permeation.

2. Discussion of the Background

Various acetals and ketals are important starting materials, intermediates, protecting groups or solvents in organic chemistry. The preparation of acetals and ketals can, according to the prior art, be carried out by reacting carbonyl compounds with alcohols or ortho-carboxylic esters in the presence of acid catalysts which may be present in homogeneous solution or as solid acidic ion exchangers (cf., for example, B. Ullmann III, 15; Houben-Weyl VI/3,3, 204; VII/1, 418, 436; D. P. Roelofsen, H. van Bekkum, Synthesis 1972, 419). Lorette et al. describe in J. Org. Chem., 1959, 1731, the influence of the stoichiometries of the starting materials and the influence of the temperature on the kinetics of this reaction. In particular, the fact is disclosed that the equilibrium of this reaction lies largely on the side of the starting materials and it is therefore not possible to achieve complete conversion of the starting materials under moderate conditions. Low temperatures down to −20° C. are necessary to achieve good conversions of starting materials and good space-time yields. In the preparation of dimethoxypropane, for example, a ratio of acetone to methanol of from 1:2 to 1:4 has to be set. Ketalization in the presence of acidic ion exchangers is described by Lorette et al. The work-up of the resulting mixture having a water content of 3-4% by weight, is made difficult by the formation of azeotropes between dimethoxypropane, water and methanol and between acetone and methanol, if the yield of dimethoxypropane is to be optimized and the losses are to be kept correspondingly low.

U.S. Pat. No. 2,827,495 describes the work-up of an aqueous/methanolic low boiler mixture by extraction with aqueous alkali, in particular sodium hydroxide solution. This process, which is carried out industrially as a countercurrent extraction, makes it possible to obtain a methanol-free, virtually pure dimethoxypropane as organic product of the extraction. However, a disadvantage is that the water content of the reaction product is relatively high.

U.S. Pat. No. 1,850,836 discloses the preparation and isolation of acetals, namely the reaction of an aldehyde with an alcohol in the presence of a catalytic amount of a mineral acid, in particular gaseous hydrogen chloride. After the reaction has reached equilibrium, the reaction mixture is neutralized with an amount of base which is at least equivalent to the acid in order to suppress the back-reaction during the work-up, and is subsequently worked up by addition of an aliphatic auxiliary solvent which is insoluble in water and forms a minimum-boiling azeotrope with the alcohol used. In this work-up, the acetal is freed of water and of most of the alcohol by means of an aliphatic solvent which is immiscible with water and the alcohol is subsequently separated off by distillation as azeotrope with the aliphatic. It is obvious that the presence of considerable amounts of aliphatic are necessary for complete removal of the alcohol, and a subsequent aqueous extraction is necessary to separate the methanol from the aliphatic solvent. Overall, the process is complicated and relatively unsuited to industrial implementation.

U.S. Pat. No. 2,837,575 describes a ketalization in the presence of gaseous hydrogen chloride. Up to 8% by weight of hydrogen chloride are used to increase the acetone conversion. This hydrogen chloride has to be subsequently neutralized with aqueous sodium hydroxide and produces a considerable quantity of salt. The subsequent work-up is carried out by complicated extractions with aqueous sodium hydroxide solutions of different concentrations and additional extraction with a volatile aliphatic hydrocarbon. A number of separation operations are required, so that the process is relatively unsuited to industrial implementation for economic reasons.

U.S. Pat. No. 4,775,447 describes a process for preparing dimethoxypropane in which an acidic heterogeneous ion exchanger is used as catalyst and an acetone:methanol ratio of from 1:1 to 1:3 is employed. However, the work-up is extraordinarily complicated and comprises, firstly, the removal of an acetone-rich azeotrope consisting of acetone, water and methanol by distillation. A corresponding amount of acetone has to be added to the remaining methanol/dimethoxypropane mixture so that an acetone/methanol azeotrope (composed of about 86% by volume of acetone and 14% by volume of methanol) is removed in a second distillation. The patent gives no further details of the separation of acetone and methanol. The separation of the water of reaction formed from the dimethoxypropane is not described.

The separation by distillation of the complex azeotrope in the water/dimethoxypropane/acetone/methanol system is described by Brunner and Scholz in Chem.-Ing.-Tech. 52 (1980), 164-166, and in Khim. Farm. Zhl. 17 (1983), 454-459. Brunner and Scholz revise the earlier results of Lorette et al. regarding the existence of a ternary azeotrope between acetone, methanol and dimethoxypropane and come to the conclusion that a) an acetone-rich azeotrope (composition as above) between acetone and methanol having a boiling point of 55.4° C. exists and that (b) a further azeotrope between methanol and dimethoxypropane having a boiling point of 61.0° C. and composed of 72.5 mol % of methanol and 27.5 mol % of dimethoxypropane exists.

The preparation of unsaturated ethers from the corresponding acetals or ketals has also been described in the literature. U.S. Pat. No. 2,667,517 discloses the elimination of alcohol in the presence of an acid catalyst selected from the group consisting of sulfonic acids in a hydrocarbon or a chlorinated hydrocarbon as solvent or diluent. In this procedure, problems are encountered as a result of high boilers which form in the reaction.

EP-A-0 197 283 describes the use of a mineral oil which is burnt after use and thus results in a considerable specific consumption of the catalyst. It is also possible to work in the absence of a solvent, as proposed in DE-A-40 39 950. Here, the ketal is generated at elevated temperatures (up to 200° C.) in the presence of a catalyst system comprising specific acids. The process is not generally applicable and has the disadvantage that contamination of the catalyst phase likewise occurs due to unselective reactions or thermal instability of the substances used, which makes discharge necessary.

In all these processes, the work-up of the product mixture by distillation or extraction is very complicated because of the azeotropes formed by methanol and water or methanol and dimethoxypropane. In addition, a back-reaction of, for example, the ketals with water can occur if no appropriate countermeasures are taken.

Furthermore, conversion and yield in particular are limited in processes involving pervaporation over unselective types of membranes, as described in EP-A-1 167 333. In these processes, large amounts of alcohol are separated off simultaneously with water, so that on a purely stoichiometric basis there is a deficiency of one of the reactants. In this case, the conversion stagnates at about 55-60% even when a plurality of membrane stages is employed. A costly work-up of the product mixture to achieve high product purity is therefore indispensable.

### SUMMARY OF THE INVENTION

It is therefore an object of the present invention to provide a process for preparing an acetal and/or ketal having a high purity in high space-time yields using very few work-up steps.

This and other objects have been achieved by the present invention the first embodiment of which includes a process for preparing an acetal or a ketal, comprising:

reacting a carbonyl compound and an alcohol in the presence of an acid catalyst and water, to obtain a reaction mixture;

removing water and alcohol from said reaction mixture in an at least two-stage pervaporation or vapor permeation process using a plurality of membranes;

feeding additional alcohol into a retentate stream of at least one of said at least two stages;

wherein at least one of said plurality of membranes in said at least two-stage pervaporation or vapor permeation process is different from the other membranes in terms of selectivity behavior in the removal of water and alcohol.

### BRIEF DESCRIPTION OF THE DRAWING

FIG. 1 shows a flowchart of the process according to the present invention.

### DETAILED DESCRIPTION OF THE INVENTION

The process according to the present invention is carried out by acetalization or ketalization in a reactor which can simultaneously be a vaporizer. The carbonyl compound is reacted batchwise or continuously with an alcohol in the presence of an acid catalyst. The water formed in the reaction and the alcohol are removed from the reaction mixture by means of an at least two-stage pervaporation or vapor permeation. Additional alcohol is fed into at least one of the stages as feed stream to the next stage. At least one of the membranes in the pervaporation or vapor permeation stages is different from the other membranes in respect of its selectivity behavior in the removal of water and alcohol.

Conversion within the meaning of the present invention refers to the reaction between starting materials to the corresponding reaction products.

The amount of alcohol which is additionally fed in is preferably 10-500 mol %, based on the carbonyl compound used. The amount of alcohol includes all values and subvalues therebetween, especially including 50, 100, 150, 200, 250, 300, 350, 400 and 450 mol %.

Water is preferably separated off by use of a membrane having a high selectivity in order to shift the reaction equilibrium to the side of the products. Thus, the progress of the reaction is accelerated. In a further stage which follows as a continuous or batchwise process step, further amounts of alcohol are fed in to maximize the reaction with the carbonyl compound. Finally, the alcohol is separated off from the acetal or ketal in a subsequent membrane stage, thus giving a very pure product without any further after-treatment.

Preferred carbonyl compounds are aldehydes of the formula RCHO or ketones of the formula R¹COR², wherein R, R¹ and R² are identical or different and are each an alkyl, alkenyl, cycloalkyl, aryl, alkylaryl or aralkyl radical having 1-15 carbon atoms, preferably 1-10 carbon atoms, and R¹ and R² may also be joined to form a ring, and the alkyl radical may also bear an acyl function having from 1 to 8, preferably from 1 to 6, carbon atoms.

Particularly preferred carbonyl compounds are formaldehyde, acetaldehyde, propionaldehyde, n-butyraldehyde and isobutyraldehyde, acrolein, benzaldehyde, formamide, N,N-dimethylformamide, acetone, methyl ethyl ketone, acetophenone, cyclohexanone, cyclododecanone, cyclodecanedione and ethyl acetoacetate.

Preferred alcohols are monohydric or polyhydric (preferably dihydric), unbranched or branched, saturated, unsaturated or aromatic alcohols having from 1 to 10, preferably from 1 to 3, carbon atoms. Particularly preferred alcohols are methanol, ethanol, n-propanol, isopropanol, allyl alcohol, benzyl alcohol, ethylene glycol, 1,3-propanediol and 1,2-propanediol.

Preferred reaction products are, in particular, formaldehyde dimethyl acetal, formaldehyde diethyl acetal, acetaldehyde dimethyl acetal, acetaldehyde diethyl acetal, 2,2-dimethoxypropane, 2,2-diethoxypropane, dimethoxycyclohexane, diethoxycyclohexane, dimethoxycyclooctane, diethoxycyclooctane, dimethoxycyclododecane and diethoxycyclododecane.

Preferred acid catalysts include acidic ion exchangers such as LEWATITE™ from Bayer AG, acidic montmorillonites, inorganic acids such as sulfuric acid, phosphoric acid and boric acid, or preferably organic carboxylic acids having from 1 to 3 carboxyl groups and from 1 to 12, preferably from 2 to 10, carbon atoms. Particularly preferred are, for example, formic acid, acetic acid, oxalic acid and 2-ethylhexanoic acid, and also, for example, branched organic acids which can be prepared by the Koch process, for example neo-octanoic, neo-nonanoic or neo-decanoic acid, and the group of sulfonic acids, e.g. methanesulfonic acid or p-toluenesulfonic acid. The acids can be used in dissolved form or as a suspension in a side stream or in the feed mixture, or else can be present in the reactor in immobilized solid form as packing, as in the case of ion exchangers or supported acid catalysts.

The process of the present invention is carried out at temperatures in the reactor or on the retentate side of from −10 to +200° C., preferably from +20 to +150° C. The reaction temperature includes all values and subvalues therebetween, especially including 0, 10, 20, 30, 40, 50, 60, 70, 80, 90, 100, 110, 120, 130, 140, 150, 160, 170, 180 and 190° C. A pressure of from 0.1 to 10 bar, preferably from 0.5 to 5 bar, is employed on the feed side. The pressure on the feed side includes all values and subvalues therebetween, especially including 1, 1.5, 2, 2.5, 3, 3.5, 4 and 4.5 bar. A pressure of from 0.1 to 1 000 mbar, preferably 10-30 mbar, is set on the permeate side. The pressure on the permeate side includes all values and subvalues therebetween, especially including 5, 10, 20, 30, 40, 50, 100, 200, 300, 400, 500, 600, 700, 800 and 900 mbar. In the process of the invention, the feed mixture is brought into contact with the membrane in liquid (pervaporation) or gaseous form (vapor permeation). Pressure and temperature on the feed side are set so that the pressure is close to the boiling pressure of the feed mixture.

The reactor can be any appropriate tank or buffer vessel. The vapors or the liquid comprising carbonyl compound, water, alcohol and, depending on the progress of the reaction, the acetal or ketal are fed directly by means of transport devices to the membrane unit in which one component is in each case separated off very selectively from this feed stream. As an alternative, the feed stream can be passed from the reactor via a separate vaporizer to the membrane module. By this procedure, alcohol is removed via the membrane in the membrane module and the depleted retentate is recirculated as vapor or in liquid form after condensation.

In the case of pervaporation, the energy for vaporization has to be supplied by the system because the component which is separated off is obtained in vapor form in the permeate, and the retentate is cooled correspondingly. Since mass transfer through the membrane is temperature-dependent or heat consumed as a result of vaporization has to be replaced, the feed stream is preferably heated by means of a preheater so as to ensure a constant optimal permeate flux. The material which has been separated off is subsequently condensed or recovered as liquid. The vacuum pump used serves first and foremost to keep the pressure (vacuum) constant and remove inert gases.

Preferred membranes are either organic polymers or inorganic materials, particularly preferably those based on ceramic. Polymer membranes are known in principle and are primarily used for dewatering but also for separation of alcohols from mixtures comprising hydrocarbons having a large number of carbon atoms, as described, for example, in EP-A-0 629 600. New developments are inorganic membranes which are used both for dewatering processes and for separations of organic compounds. These ceramic membranes preferably have a selective, defect-free zeolite layer which has selective separation properties because of its uniform crystal structure.

In the membrane module, alcohol and water are separated off selectively from the carbonyl compound membrane types operating in various ways. The correspondingly depleted retentate is condensed and can be returned to the reactor and the membrane module where it can be taken off as bottoms in batch or continuous operation.

According to the present invention, the mixture is fed to a further reactor stage in which the same alcohol as that used as starting material in the first stage is fed in. As a result the conversion of the carbonyl compound is completed. However, the alcohol separated off together with the water as permeate can, if required, be separated off from the water by a simple distillation apparatus and be recirculated to the reaction as starting material.

In a preferred embodiment, use is made of multilayer membranes, i.e. membranes which comprise a carrier layer, a porous support layer and an actual separation layer. Preferred carrier layers are flexible woven fabrics made of fibers, including woven metal meshes, polyesters, polyetheramides, carbon fibers, etc., for example membranes of the PERVAP type from SULZER-CHEMTECH or NaA zeolite membranes from MITSUI. Inorganic materials having porous structures, e.g. ceramics, are likewise suitable.

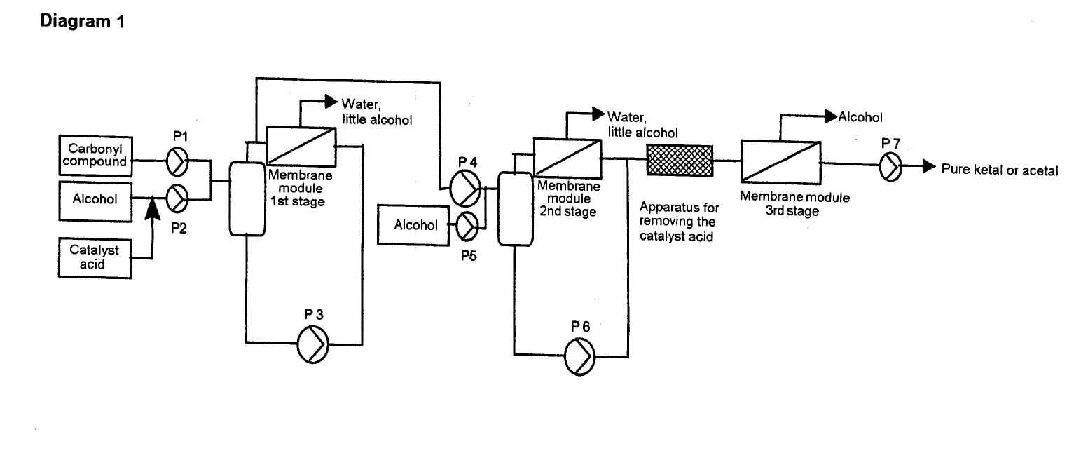

According to the present invention, the pervaporation is carried out in a plurality of stages. In a first stage, the carbonyl compound and the appropriate alcohol are reacted in the reactor which is provided with a bypass line into which the membrane module for separating off water is integrated. This step can be carried out batchwise, but is advantageously carried out continuously. In a continuous operating mode, carbonyl compound and alcohol are metered in as feed stream via the pumps P1 and P2 (see FIG. 1) in an amount corresponding to the total amount of product discharged via P4 and water and alcohol removed in the membrane modules 1, 2 and 3. The stages are carried out using membrane types which separate off large amounts of water, i.e. make a high flux possible. However, a number of membrane types operate less selectively at high fluxes, so that relatively large amounts of alcohol are also separated off.

In the cascade mode described, the conversion of carbonyl compound is very high, as a result of which the excess alcohol used has to be separated off from the acetal or ketal in the last membrane stage. In this last stage of the cascade, use is made of a membrane type which allows large mass flows of alcohol but selectively holds back the hydrophobic acetals or ketals (for example NaY zeolite membranes). To avoid back-reaction of the ketal or acetal to reform alcohol in the last stage, the catalyst acid can advantageously be separated off at low temperatures as relatively high-boiling bottoms in a distillation carried out at low temperatures and short residence times in a falling film or thin film evaporator. The alcohol which has been separated off can be reused without further work-up in the synthesis to form the acetal or ketal.

The process of the present invention has significant advantages over conventional processes for preparing acetals and ketals. In particular, high reaction rates and selectivities in the reaction can be achieved at relatively low temperatures. The feed stream can be fed directly either in liquid form or as vapor to the membrane module.

The removal of the water is absolutely necessary for progress of the reaction and thus for the conversion to occur quickly. Water and relatively large amounts of alcohol, in particular methanol, are separated off at an increased flux through a customary hydrophilic membrane type. The product remaining in the reactor toward the end of the reaction already has a very high proportion of ketal or acetal. When homogeneously dissolved acids are used as catalyst, the acid has to be separated off by distillation or extraction before the last membrane cascade in order to isolate the desired acetal or ketal in highly pure form. In this case, a work-up by distillation using a small number of theoretical plates in a thin film evaporator or short path evaporator is often sufficient.

Having generally described this invention, a further understanding can be obtained by reference to certain specific examples which are provided herein for purposes of illustration only, and are not intended to be limiting unless otherwise specified.

### EXAMPLES

A mixture of 348 g of acetone, 576 g of methanol and 5 g of neo-decanoic acid (Versatic 10 from Resolution) was placed in a pervaporation module for experiments in vapor permeation operation comprising a reaction vessel having a volume of 1 500 ml and a membrane section having a membrane area of about 0.1 m². The mixture was heated to 60° C. while stirring well. The saturated vapor was conveyed from the upper end of the reaction vessel to the upper end of the membrane cell and flows past the membrane from the top downward. In the flow direction, the retentate vapor was condensed in a condenser and recirculated to the reaction vessel by means of a circulation pump. The permeate passing through the membrane was collected and condensed in the permeate condenser. A pressure of from 2 to 10 mbar absolute was employed on the permeate side. The condensate comprised water and methanol in a ratio of about 1:0.5. Under these conditions, a flux of about 1 kg/m² h was achieved in the dewatering by means of a PERVAP 2210 or 2510 membrane from SULZER CHEMTECH. The conversion to dimethoxypropane in the first stage was about 55%. The retentate stream, comprising 343 g of dimethoxypropane, 245 g of methanol and 156 g of acetone, was fed batchwise or continuously to a further stage of a membrane module. 120 g of methanol were added to 744 g of this retentate mixture from the first stage and the resulting mixture was once again subjected to treatment under conditions comparable to those in the first stage. 168 g of a mixture of methanol and water in a ratio of 1:3 were then taken off as permeate. The retentate comprised 618 g of dimethoxypropane, about 1 g of acetone and 295 g of methanol. The conversion to dimethoxypropane was about 99%. This mixture was then fed to a further stage for treatment in a membrane unit, in which 290 g of methanol were removed from this feed mixture by means of another type of membrane from SULZER CHEMTECH, viz. PERVAP 2256, at a membrane flux of about 6 kg/m² h.

The retentate then comprised a mixture of 616 g of dimethoxypropane and 4 g of methanol, so that the target product was present in the retentate in a concentration of 98.4%.

German patent application 10218916.1 filed Apr. 27, 2002, is incorporated herein by reference.

Numerous modifications and variations on the present invention are possible in light of the above teachings. It is therefore to be understood that within the scope of the appended claims, the invention may be practiced otherwise than as specifically described herein.

What is claimed is:

1. A process for preparing an acetal or a ketal, comprising:
reacting a carbonyl compound and an alcohol in the presence of an acid catalyst and water, to obtain a reaction mixture;
removing water and alcohol from said reaction mixture in an at least two-stage pervaporation or vapor permeation process using a plurality of membranes;
feeding additional alcohol into a retentate stream of at least one of said at least two stages;
wherein at least one of said plurality of membranes in said at least two-stage pervaporation or vapor permeation process is different from the other membranes in terms of selectivity behavior in the removal of water and alcohol.

2. The process as claimed in claim 1, wherein the additional alcohol is fed in downstream of a first pervaporation or vapor permeation stage of said at least two stages.

3. The process as claimed in claim 1, wherein an amount of said additional alcohol is 10-500 mol %, based on a total amount of said carbonyl compound.

4. The process as claimed in claim 1, wherein said carbonyl compound is a) an alddhyde of the formula RCHO or a b) ketone of the formula R¹COR²;
wherein R, R¹ and R² are each identical or different and are each an alkyl having having 1-15 carbon atoms, an alkenyl radical having 2-15 carbon atoms, a cycloalkyl radical having 3-15 carbon atoms, an aryl radical having 6-15 carbon atoms, an alkylaryl or aralkyl radical each having 7-15 carbon atoms;
wherein R¹ and R² may be joined to form a ring; and
wherein said alkyl radical optionally has an acyl group having from 1 to 8 carbon atoms.

5. The process as claimed in claim 4, wherein said carbonyl compound is selected from the group consisting of formaldehyde, acetaldehyde, propionaldehyde, n-butyraldehyde and isobutyraldehyde, acrolein, benzaldehyde, formamide, N,N-dimethylformamide, acetone, methyl ethyl ketone, acetophenone, cyclohexanone, cyclododecanone, cyclodecanedione, ethyl acetoacetate and mixtures thereof.

6. The process as claimed in claim 1, wherein said alcohol is a monohydric or polyhydric, unbranched or branched, saturated, unsaturated or aromatic alcohol having from 1 to 10 carbon atoms.

7. The process as claimed in claim 2, wherein said alcohol is selected from the group consisting of methanol, ethanol, n-propanol, isopropanol, allyl alcohol, benzyl alcohol, ethylene glycol, 1,3-propanediol, 1,2-propanediol and mixtures thereof.

8. The process as claimed in claim 1, wherein said acid catalyst is dissolved or suspended in said reaction mixture or is present in immobilized form as solid packing.

9. The process as claimed in claim 8, wherein said acid catalyst is an organic carboxylic acid having from 1 to 3 carboxyl groups and from 1 to 12 carbon atoms.

10. The process as claimed in claim 8, wherein said acid catalyst is selected from the group consisting of formic acid, acetic acid, oxalic acid, 2-ethylhexanoic acid, neo-octanoic acid, neononanoic acid, neodecanoic acid, methanesulfonic acid, p-toluenesulfonic acid and mixtures thereof.

11. The process as claimed in claim 1, wherein said catalyst is a solid and is located in packing or is suspended in a side stream or in the feed mixture.

12. The process as claimed in claim 1, wherein a temperature in the reactor is from −10 to +200° C.

13. The process as claimed in claim 1, wherein a temperature in the reactor is from +20 to +150° C.

14. The process as claimed in claim 1, wherein a pressure is a) from 0.1 to 10 bar on the feed side of the pervaporation or vapor permeation stages and b) from 0.1 to 1 000 mbar on the permeate side.
